(19) United States
(12) Patent Application Publication
(10) Pub. No.: US 2010/0098220 A1
(43) Pub. Date: Apr. 22, 2010

(54) Intra-oral envelope blank for a silver-based x-ray film

(76) Inventor: Dennis W. Allmer, Yaphank, NY (US)

Correspondence Address: CLIFFORD G. FRAYNE, 136 DRUM POINT RD, SUITE 7A, BRICK, NJ, 08723, US

(21) Appl. No.: 12/288,295

(22) Filed: Oct. 20, 2008

(51) Int. Cl. A61B 6/14

(52) U.S. Cl. 378/169

(57) ABSTRACT

There is disclosed an intra-oral x-ray envelope blank for a silver-based x-ray film chip formed of a light opaque front panel member comprised of a base panel portion and an upper enclosure panel portion and a back radiation opaque panel member heat sealed to the base panel portion of the front panel member thereby forming an x-ray receiving film pocket and wherein the upper enclosure panel portion is subsequently folded over the back panel member and sealed thereto and wherein said outer surface of the upper enclosure panel portion include debossed or material relieved slits providing tear directions for accessing the x-ray receiving film chip pocket.

### BACKGROUND OF THE INVENTION

1. Field of the Invention

This invention relates to radiographic imaging, and more particularly to an envelope blank for encapsulating a silver-based x-ray film chip.

2. Description of the Prior Art

Dental intra-oral x-ray films have been used for many years and generally consist of a film chip encapsulated in a light tight envelope or packet formed of PVC or polyolefin elastomeric material. Generally, an intra-oral x-ray film envelope is comprised of three components including the x-ray film chip, a paper wrap, and a radiation shielding member.

Health care givers and dental technicians are trained in the application and use of such film with a sequence use typical for all embodiments. Initially, the x-ray envelope is positioned within the oral cavity and exposed to a source of x-ray radiation on a side formed of radiation pervious material whereupon the x-ray envelope is removed from the oral cavity and taken to a light secure environment, such as a dark room or daylight loader. The x-ray envelope is thereafter opened for the unobstructed removal of the exposed x-ray film chip.

Current dental intra-oral x-ray film envelopes or packets generally present problems in difficulty in the facile opening and accessing of the exposed x-ray film chip. The x-ray film chip is sensitive to any form of pressure and will and does sustain damages when subjected to stressful handling thereby effecting the medical or dental evaluation and diagnosis, and oftentimes requires the retaking of the x-rayed region. Such difficulties include the imposing of artifacts on the x-ray film material or film chip during the opening process must be factored into the evaluation and review of the resulting diagnostic radiography.

### OBJECTS OF THE INVENTION

An object of the present invention is to provide an intra-oral x-ray envelope blank for x-ray film chips of a silver-based substrate permitting of facile opening capabilities.

Another object of the present invention is to provide an intra-oral x-ray envelope blank for x-ray films of a silver-based substrate permitting of facile opening capabilities thereby to minimize potential damage to the exposed x-ray film chip.

Still another object of the present invention is to provide an intra-oral x-ray envelope blank for x-ray film chips of a silver-based substrate permitting facile opening capabilities thereby ensuring an artifact free exposed x-ray film chip.

### SUMMARY OF THE INVENTION

These an other objects of the present invention are achieved by an intra-oral x-ray envelope blank for a silver-based x-ray film chip formed of a light opaque front panel member comprised of a base panel portion and an upper enclosure panel portion and a back radiation opaque panel member heat sealed to the base panel portion of the front panel member thereby forming an x-ray receiving film chip pocket and wherein the upper enclosure panel portion is folded over the back panel member and sealed thereto and wherein said outer surface of the upper enclosure panel portion include debossed or material relieved slits providing tear directions for accessing the x-ray receiving film chip pocket.

### BRIEF DESCRIPTION OF THE DRAWINGS

These and other objects of the present invention will become more readily apparent from the following detailed description thereof, particularly when taken with reference to the accompanying drawings, wherein:

FIG. 1 is an elevational view of the radiation exposure side of an dental intra-oral envelope blank for a silver-based x-ray film chip of the present invention;

FIG. 2 is an elevational view of the opposite side of the dental intra-oral x-ray envelope blank for a silver-based x-ray chip of the present invention;

FIG. 3 is a cross-sectional view taken along the lines II-II of FIG. 2 of the intra-oral x-ray envelope blank of the present invention; and

FIG. 4 is a cross-sectional view of the envelope blank of the present invention encapsulating, inter alia, a silver-based x-ray film chip.

### DETAILED DESCRIPTION OF THE PRESENT INVENTION

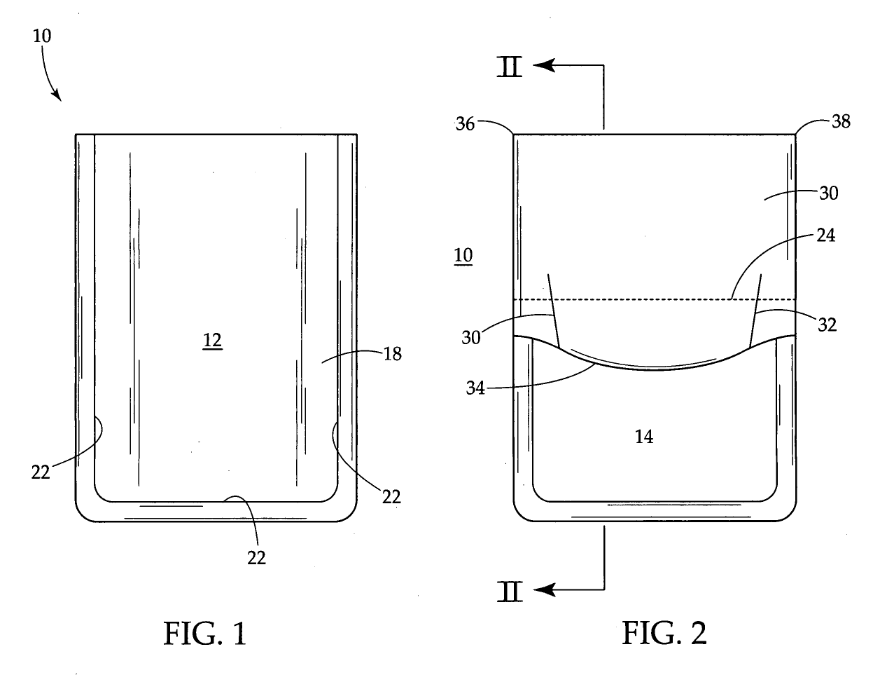

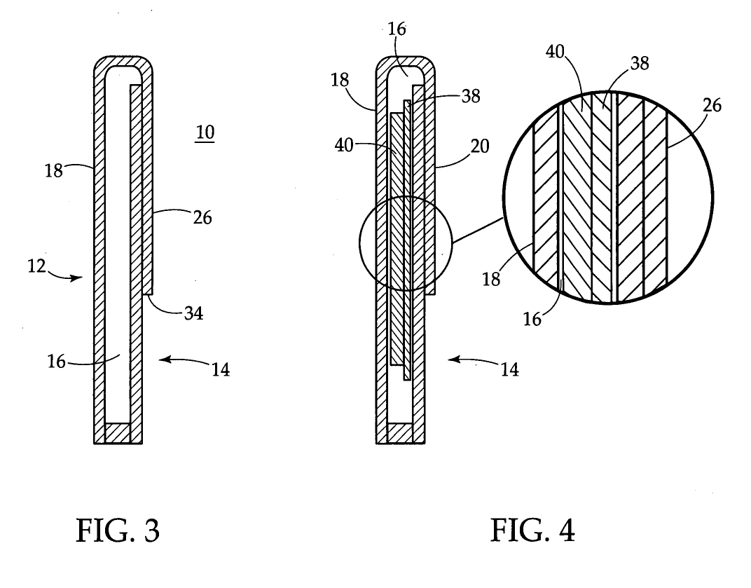

Referring now to drawings, and particularly FIGS. 1 to 3, there is illustrated a dental intra-oral x-ray envelope blank, generally indicated as 10, comprised of a front panel member and a rear panel member, generally indicated as 12 and 14, respectively, forming a pocket 16 sized to accommodate intra-oral dental x-ray film chips of ISO sizes #0 (35 mm×22 mm), #1 (40 mm×24 mm), #2 (41 mm×31 mm or 41 mm×30 mm), #3 (54 mm×27 mm), and #4 (76 mm×57 mm). The front panel member 12 is formed of a radiation penetrable, light opaque thermoplastic material. The rear panel member 14 is formed of a light opaque thermoplastic material and may also include radiation opaque material, as more fully hereinafter discussed.

The front panel member 12 is comprised of a base panel portion 18 and an upper enclosure panel portion 20 wherein the base panel portion 18 is heat sealed 22 to the rear panel member 14 about peripheral bottom and side edges, referring particularly to FIG. 1, thereby forming the x-ray film receiving pocket 16. The upper panel portion 20 is provided with a pressure sensitive adhesive 24, illustrated by the dashed lines in FIG. 2, deposited on a side to be positioned on the rear panel member 16. On an outer side 26 of the upper panel portion 18, there are formed cooperating partially embossed directional slits 30 and 32 originating at a free end 34 of the upper panel portion 20 and directed towards end folded over portions 36 and 38 of the front panel member 12, referring particularly to FIG. 2.

The directional slits 30 and 32 formed on the outer side 26 of the upper panel portion 20 comprise only about 25 percent of material thickness. Such material thickness represents a functional attribute of balance between retaining light seal and integrity of the x-ray film chip to be positioned in the thereby to facilitate the mechanism for opening the intra-oral envelope after exposure so as to provide dimensional consistency for access to the contents of the x-ray receiving pocket 16, as more fully hereinafter discussed.

In one usage configuration, the rear panel member 14 is formed of a radiation opaque material in addition to possessing the property of being light opaque. To effect such radiation opaque property, there is included in the thermoplastic material for the rear panel member 18 of the x-ray envelope blank 10, a metal in the form of a non-lead metal fiber powder, such as tungsten or a stainless steel alloy powder. In such usage configuration, an x-ray film or chip is positioned in the pocket 16 of the envelope blank 10 and used in the manner as hereinabove discussed with regard to standard practice.

In another usage configuration wherein the thermoplastic material of the rear panel member 14 does not possess radiation opaque properties, a shielding member 38 together with the x-ray film or chip 40 is positioned within the pocket 18 between the film chip 40 and rear panel member 16, referring particularly to FIG. 4. The shielding member 38 may be in the form of a lead foil or like radiation opaque material.

In use, after exposure of selected area of the oral cavity of the user and removal thereof, access to the exposed x-ray film or chip is effected by gripping the free end of the upper enclosure portion 20 of the front panel member 12 and pulling upwardly whereupon the sealing slits 30 and 34 facilitate the tearing of such upper enclosure portion 20 across the overlap seam or sealing 24 towards the perimeter side edges 36 and 38 of the x-ray film chip envelope blank 10. The starting slits 30 and 32 require less force in opening the film packet and a more consistent dimension of opening for internal access. Still further, the requirement of less force to open the film packet substantially eliminates user imposed artifacts from being imposed on the film chip.

While the present invention has been described with reference to an exemplary embodiment thereof, it will be understood to those of ordinary skills that many modifications are apparent; and that this application is intended to cover any adaptations or variations thereof. Therefore, it is manifestly intended that this invention be only limited by the claims and the equivalents thereof.

What is claimed is:

1. An envelope blank for a silver based x-ray film, which comprises:
a front panel member comprised of a base panel portion and an upper enclosure panel portion, an outer surface of said upper enclosure panel portion being formed with material relieved slits; and
a rear panel member, said rear panel member sealed to said front panel member thereby forming an x-ray film chip receiving pocket, said upper enclosure panel portion of said front member capable foldable over said rear panel member after positioning a silver-based x-ray film chip in said x-ray film chip receiving pocket and sealed thereto for intra-oral use.

2. The envelope blank for a silver based x-ray film chip as defined in claim 1 wherein said material relieved slits providing tear directions of said upper enclosure panel portion to provide access to said x-ray receiving pocket.

3. The envelope blank for a silver based x-ray film chip as defined in claim 2 wherein said material relieved slits are pointed towards end folds between said lower front panel portion and said upper enclosure panel portion.

4. The envelope blank for a silver based x-ray film chip as defined in claim 1 wherein said front panel member is formed of a radiation permeable, light opaque thermoplastic material.

5. The envelope blank for a silver based x-ray film chip as defined in claim 4 wherein said rear panel member is formed of a radiation and light opaque thermoplastic material.

6. The envelope blank for a silver based x-ray film chip as defined in claim 5 wherein radiation opaque properties are provide by a metal powder in the form of a non-lead metal fiber powder incorporated into said thermoplastic material.

7. The envelope blank for a silver based x-ray film chip as defined in claim 4 wherein said rear panel member is heat sealed to said lower panel portion of said front panel member about the bottom and sides edges thereof thereby forming said x-ray film chip receiving pocket.

8. An intra-oral x-ray film packet, which comprises;
a front panel member comprised of a base panel portion and an upper enclosure panel portion, an outer surface of said upper enclosure panel portion being formed with material relieved slits;
a rear panel member, said rear panel member sealed to said front panel member thereby forming an x-ray film chip receiving pocket, said upper enclosure panel portion of said front member capable foldable over said rear panel member;
an x-ray shielding member disposed in said x-ray film chip receiving pocket; and
a silver-based x-ray film chip disposed in said x-ray film chip pocket between said front panel member and said x-ray shielding member juxtaposed to the rear panel member.
